(19) United States
(12) Patent Application Publication
(10) Pub. No.: US 2020/0397366 A1
(43) Pub. Date: Dec. 24, 2020

(54) Sleep Activity Detection Method And Apparatus

(71) Applicant: Babylon Partners Limited, London (GB)

(72) Inventors: Alessandro Rodolfo GUAZZI, London (GB); Maxim OSIPOV, London (GB)

(21) Appl. No.: 16/975,760

(22) PCT Filed: Feb. 26, 2019
(86) PCT No.: PCT/GB2019/050532

§ 371 Date: Aug. 26, 2020

(30) Foreign Application Priority Data
Feb. 26, 2018 (GB) 1803102.1

(51) Int. Cl. A61B 5/00; A61B 5/0205

(52) U.S. Cl. CPC A61B 5/4812; A61B 5/7235; A61B 5/02055; A61B 5/02405; A61B 2560/0247; A61B 2560/0252; A61B 2560/0257; A61B 2562/0219

(57) ABSTRACT

A method and apparatus for detecting sleep state from both physiological and environmental sensors. The analysis is performed on segmented signals and sleep state is detected if sleep probability is above a predefined threshold. The method may be applied to any noisy signal or set of signals acquired from a user and environment in which there is uncertainty about the presence of sleep state and in real-time analysis of signals, preceding further analysis of the sleep signal.

### BACKGROUND

Conventional methods for detecting sleep activity are traditionally based on physiological data acquired directly from subjects, such as using polysomnography (typically including EEG, EOG, EMG, ECG and actigraphy signals, as well as others or a subset of those), and using thresholds or classifiers to distinguish between the wakefulness and sleep states on fixed intervals (for example minute-by-minute). In ambulatory settings these methods are affected by data missingness and confounding factors, such as user compliance to wearing sensors.

### TECHNICAL FIELD

The present invention relates to the detection of sleep state using both physiological and environmental sensors in “free-living” conditions on both fixed and variable-length intervals. In more detail, it relates to the analysis of sensor signals to determine which sections of the signals contain episodes of sleep. Being able to detect episodes of sleep through the analysis of signals, not necessarily acquired directly from the subject, is advantageous as it solves the user compliance problem and can allow further sleep analysis to be performed and long-term patterns of sleep identified.

### SUMMARY

Accordingly, the present invention provides a method of detecting and displaying episodes of sleep, comprising the steps of: acquiring one or more input signals from a physiology sensor, a physical activity sensor or an environment sensor which may be related to activities of wakefulness or sleep (sleep evidence), normalizing the signals received from at least one of the sensors using a personalised dynamic range based on circadian signals variation, identifying continuous segments in the input signal(s) received from the sensors, inferring a personalised physiological model of circadian rhythm, inferring a personalised model of sleep/wakefulness pressure, combining the normalised input signal(s), the segments, the circadian rhythm model and the sleep/wakefulness pressure model to infer sleep state.

Physiological signals may include heart rate and derivatives, such as heart rate variability (HRV) measures, skin conductance and derivatives, such as electrodermal response measures, and body temperature, as well as other signals representing bodily processes.

The physical activity sensor(s) output signals may include acceleration, rotation, orientation, geolocation and speed data, as well as other signals related to measurement of physical motion of a user.

The environmental sensor(s) output signals may include sound, light, ambient air temperature and humidity, atmospheric pressure data, as well as other signals related to user's environment.

The input signal may be a pre-recorded signal, or the digital signal produced by a sensor in real time. Examples of the latter are the signals obtained from sensors present on phones, headsets, computers, smart-watches, and household appliances. Thus, the sleep activity detection may be used in real-time if a person is wearing, holding or in the range of a sensor while the sensor is recording.

Thus, the input signal can be a time series of sensor(s) data, and it can comprise of several channels from different sensors operating on one or several recording devices. If multiple sensors are present each sensor can be treated individually or in combination with other sensors.

The normalisation of signals may for example include elimination of bias and outliers and scaling the signal to a specific range (for example [0 . . . 1]) from the original range of signal empirically identified from N observations (where N may for example cover 3 days of observations), and performing such scaling on each M signal observations and using the directly preceding N observations to calculate the range.

The identification of continuous segments (i.e. sequences of observations within which the signal of interest is stationary) in the signal may be performed using a change point detection method, such as Bayesian Online Change Point Detection or any other suitable method, where signals can be considered individually or as a combined multidimensional vector. If considered individually or as a set of multidimensional vectors, the segments derived from different vectors can be combined into a single segmentation where each individual change point of each individual signal or a set of signals is considered as a change point in the combined segmentation.

The circadian rhythm model defines the individual probability of sleep if driven solely by the circadian pacemaker, i.e. with approximately 24-hour period where deviations from this period, as well as the shape of the function over time, are user-specific parameters and can be identified from a physiological model or user behaviour. The example circadian rhythm model can be represented for example by the Gaussian Mixture Model where peaks of the mixture components are positioned at 3 AM and the standard deviation of components is 2 hours. The sleep-wake pressure model can also be described as Gaussian Process or a periodic function with fixed or variable period and amplitude.

The sleep-wake pressure model defines the individual probability of sleep if driven solely by sleep and wakefulness times, i.e. with the sleep probability increasing after a prolonged period of wakefulness and wakefulness probability increasing after a prolonged period of sleep. The rate of change of such pressure, as well as other parameters of the function are user-specific parameters and identified from a physiological model or user behaviour. An example sleep-wake pressure model can be represented for example by a piecewise linear function, comprised of minimal-pressure (at least 8 hours of sleep within the last 16 hours), increasing-pressure (8 to 12 hours of activity in the last 16 hours), maximum-pressure (12 to 16 hours of activity in the last 16 hours) and decreasing-pressure (4 to 8 hours of sleep in the last 8 hours) segments. The sleep-wake pressure model can also be expressed as Lotka-Volterra equations or a function of melatonin sleep hormone secretion and a function of other sleep-related processes in a body.

The invention may be embodied in a computer program comprising program code means for executing on a computer system the method steps. Alternatively, it can be embodied in firmware or hardware, or as program code for computing devices such as smartphones, smart-watches or household appliances.

Another aspect of the invention provides an apparatus for analysing an input signal to detect the presence of human sleep activity in the signal, the apparatus comprising an input for receiving the input signal, a processor for processing the input signal, wherein the processor is configured to execute the method above. The apparatus further includes an output to output the ratio or the signal detected, for example by means of a display. The apparatus further comprises memory (e.g. non-tangible memory) configured to store the computer program described herein. In one example, the method described herein is a computer-implemented method.

### BRIEF DESCRIPTION OF THE DRAWINGS

The invention will be further described by way of example with reference to the accompanying drawings in which:

FIG. 1 is a flow diagram that illustrates the method of one embodiment of the invention;

FIGS. 2A to 2E show the results of applying the embodiment of FIG. 1 to an intermittent day-long sensors recording; and

FIG. 3 is a schematic illustrating the sleep activity detection apparatus according to an embodiment of the invention.

### DETAILED DESCRIPTION

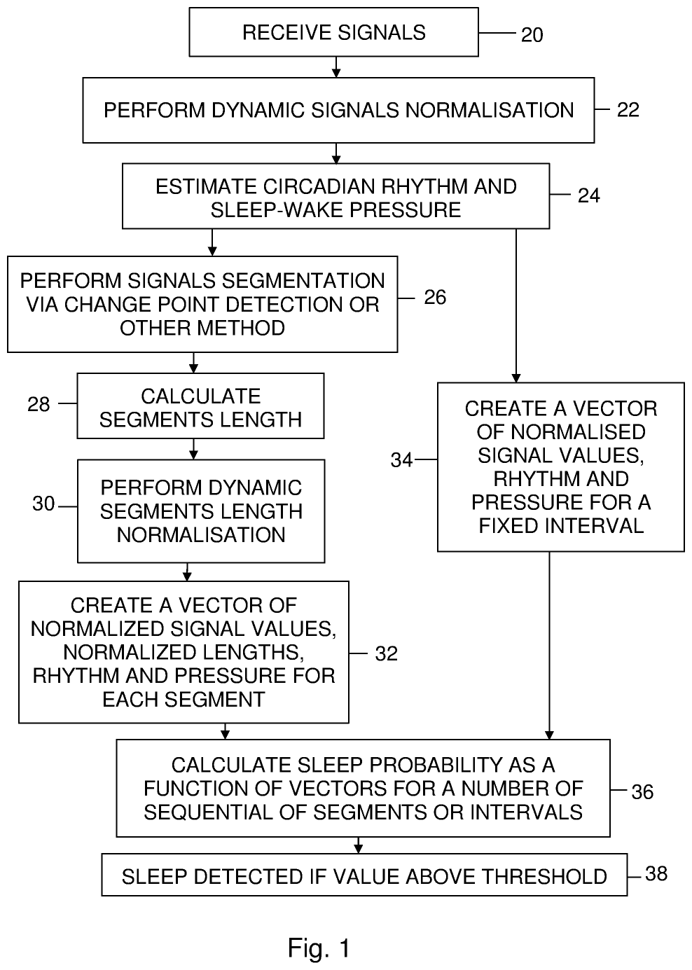

FIG. 1 is a flow diagram illustrating the application of an embodiment of the invention to the analysis of a number of signals derived from a sensor or a plurality of sensors.

In step 20 digital signals from a sensor or sensors collect information of physical activity of a user and environmental information, for example light and sound, at a sampling rate fₛ of 1/60 Hz.

In step 22, the signal is normalised to a [0 . . . 1] range. The signal may be normalised, for example, by subtracting a minimum and dividing by a maximum of a predefined period of the signal, such as the last 3 days of observations. Different normalisation strategies can be used.

In step 24, a circadian rhythm value is calculated for example using the following cardiac rhythm model:

r=N(t,μ,σ²)/c_{N}

In this formula, N is the Gaussian distribution, t is the current time in milliseconds (expressed as offset from the nearest midnight time, thus in the range of −12 to +12 hours), μ is the rhythm peak time closest to the time t (for instance, the periodic rhythm peak may occur at 3 AM every 24 hours), σ² is the standard deviation of N (e.g. the rhythm “peakiness”, selected so that the function returns approximately 0.5 at +/−4 hours from the rhythm peak) and C_{N} is the normalization coefficient to ensure that the value of r equals 1 at t=μ. Other rhythm functions and inputs signals can (in addition to time) be used to produce the circadian rhythm model output.

In step 24, a sleep-wake pressure value is calculated, for example using the following press-awake pressure model:

if 4<s₈<8:

p=(8−s₈)/4

else if s₁₆>=8:

p=0

else if 8<w₁₆<=12:

p=(w₁₆−8)/4

else if 12<w₁₆<=16:

p=1

s₈ and s₁₆ are estimated or measured sleep times in the last 8 and 16 hours respectively (in hours), which represent the estimated or measured times during which the user has been asleep. w₁₆ is the wakefulness time in the last 16 hours (in hours), which represents the estimated or measured time during which the user has been awake in the last 16 hours. Other sleep-wake pressure functions and input signals can be used to produce the sleep-wake pressure model output. Each of the values for s₈, s₁₆ and w₁₆ may be a pre-set estimate or a value measured by observation for the first iteration of the algorithm. Subsequent iterations of the algorithm may rely on the outputs of the algorithm to determine the values for s₈, s₁₆ and w₁₆.

At this point, the method may proceed with steps 26 to 32 in which calculations are carried out to perform segmentation of signals and infer characteristics of each segment. Alternatively, the method may proceed with step 34 in which fixed segments are calculated, and characteristics of the fixed segments are determined, such as the mean and standard deviation values of the signals for each segment.

In step 26, a change point or segmentation method such as for example the Bayesian Online Change Point Detection (implemented according to “Bayesian online changepoint detection” by Adams and MacKay, which is accessible at https://arxiv.org/abs/0710.3742) is applied to signal, for example with parameters μ=0.0, κ=0.1, α=0.1, β=0.1 and the expected run length 60 (minutes), and the lengths of resulting segments calculated in step 28.

In step 30, the lengths of segments are dynamically normalised by dividing by the maximum length of segment detected in a period, such as the last N (for example N equals 3) days.

In step 32, segment-specific feature vectors are created, including but not limited to mean and standard deviation values of signal for each segment and including these values and (1/normalized segment length)'s calculated on step 30 into the resulting feature vectors.

In step 36, a sleep probability value is calculated. The sleep probability value may be calculated using the equation:

s=S(a₁v₁+a₂v₂+ . . . +a_{N}v_{N},x₀)

In this equation, S is a logistic function S(x)=1/(1+e^{-5(x-x}^{₀}⁾). The values each represent one of the signals features calculated previously (for example mean, standard deviation and 1/(normalized segment length)). The values a_{1 . . . N}, are the feature weights for each signal, for example each equal to 1/N. The value x₀ is the x-value of the midpoint of the logistic function. In this example, x₀=b₁r+b₂p, where r is a circadian rhythm value output from the circadian rhythm model at the current time, p is a sleep-wake pressure value output from the sleep-wake pressure model and b_{1,2} are the individual weights of these values (0.5 or other values between 0 and 1). Other sleep probability functions and signal features can be used to calculate the sleep probability.

In step 38, sleep is detected by comparing the sleep probability to a pre-defined threshold. As an example, the pre-defined threshold is chosen to be 0.5. The threshold may change depending on the level of sensitivity and specificity desired for the application and the context of the user. Multiple thresholds can be set to distinguish between sleep stages.

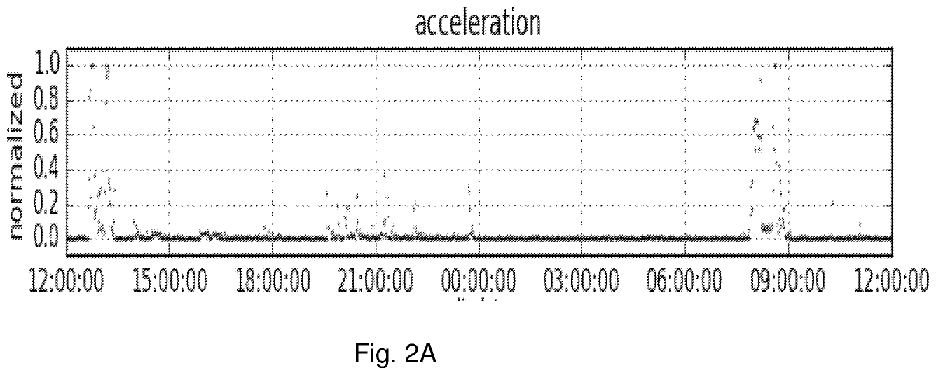

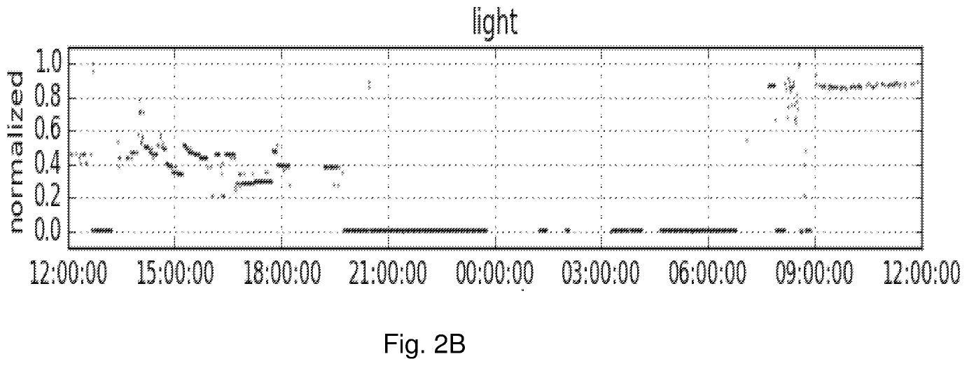

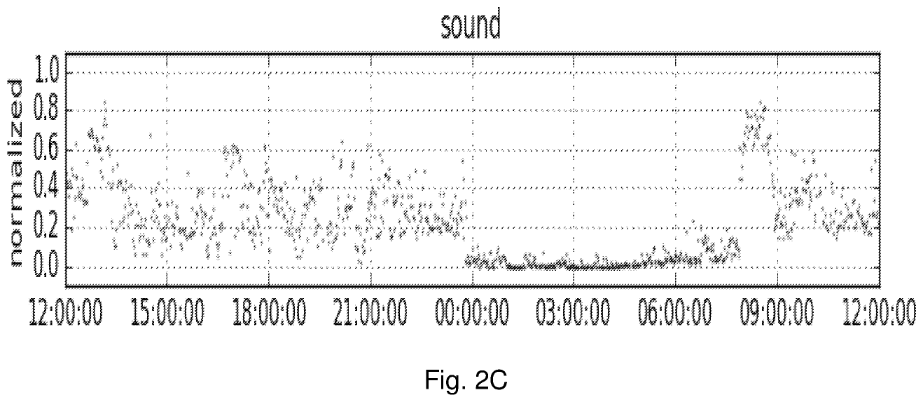

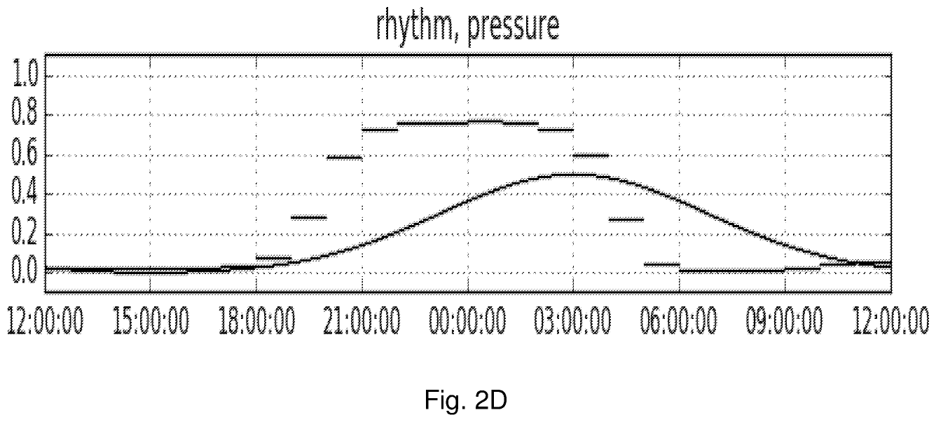

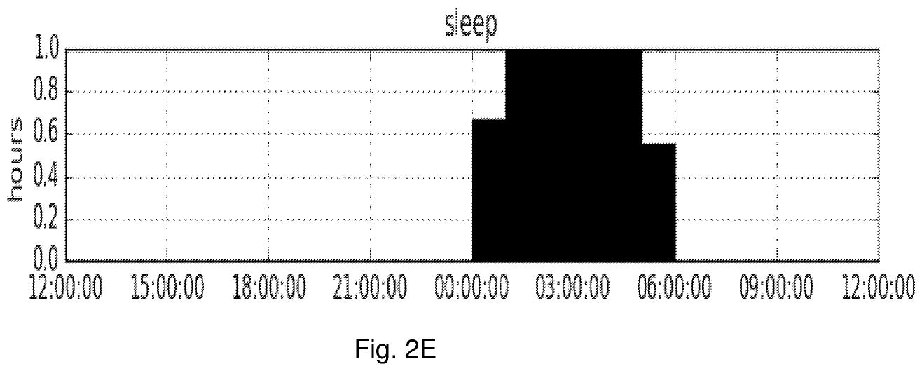

FIGS. 2A to 2E show the results of applying this example of the invention to a day-long (or 24-hour period) recording carried out using a smartphone, and its sensors. FIGS. 2A to 2C display the chosen signals measured by the smartphone (acceleration, light and sound measured by the smartphone's gyroscope, camera and microphone respectively) dynamically normalised to a range of 0 to 1. FIG. 2D displays the values of output from the circadian rhythm model and from the sleep pressure model. FIG. 2E displays the sleep time as estimated by the algorithm. As can be seen, the invention is suitable for near-real-time analysis over long periods of time using relatively few resources and incomplete information.

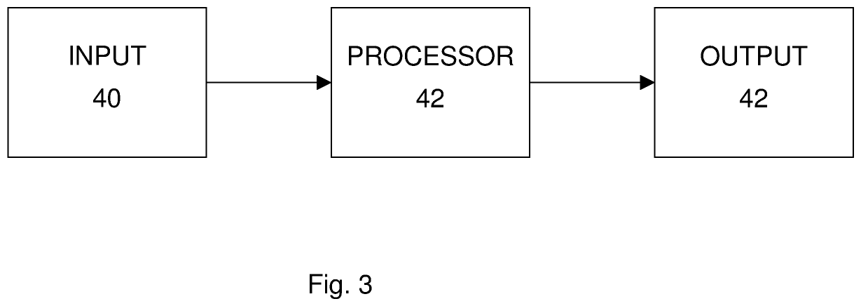

An embodiment of this invention may be a computer program for running on a computer system which receives the input sensors data time series and estimates the result of the sleep detection. The sleep activity may also be displayed. Alternatively the invention may be incorporated into a dedicated apparatus, such as a monitor, which is shown as a schematic in FIG. 3. The apparatus comprises an input 40 for receiving the input data, a process 42 for executing the processing steps described above, and an output 44, for example a display, for outputting the detection of the sleep. Alternatively, the output may feed the segmented data, sleep rhythm and pressure values for further processing into other methods, for example a sleep stages identification method.

1-32. (canceled)

33. A method for detecting sleep activity, the method comprising:
receiving one or more input signals;
performing normalisation of the one or more input signals;
calculating a circadian rhythm value from a circadian rhythm model and a sleep-wake pressure value from a sleep-wake pressure model;
performing segmentation of the normalised one or more input signals;
calculating a sleep probability value as a function of at least one characteristic of the segmented normalised one or more input signals, the calculated circadian rhythm value and the calculated sleep-wake pressure value; and
outputting an output signal indicating that the one or more input signals comprises a sleep episode, if the sleep probability value is above a predefined sleep detection threshold.

34. The method according to claim 33 wherein the one or more input signals are acceleration, light and sound signals.

35. The method according to claim 33 wherein the segmentation is performed by applying the Bayesian Online Change Point Detection method to the one or more input signals.

36. The method according to claim 33 wherein outlier detection and elimination is performed before the normalisation is performed.

37. The method according to claim 33 wherein the one or more input signals are pre-processed to extract a specific characteristic of the signal which is in turn used as an input signal.

38. The method according to claim 33 wherein the sleep detection threshold is determined based on a desired sensitivity and/or specificity.

39. The method according to claim 33 wherein the input signals are combined using a weighted average function to calculate the sleep probability.

40. The method of claim 39, wherein the combined input signals were processed using a logistic function.

41. The method according to claim 40 wherein the midpoint of the logistic function is a function of the circadian rhythm value and the sleep-awake pressure value.

42. The method according to claim 40 wherein the midpoint of the logistic function is based on a weighted average of the circadian rhythm value and the sleep-awake pressure value.

43. The method according to claim 33 wherein the one or more input signals consist of time series of digital sensor data from any of a heart rate sensor, a heart rate variability (HRV) sensor, a skin conductance sensor and derivatives thereof; an acceleration sensor, a rotation sensor, an orientation sensor, a geolocation sensor and a speed sensors; a sound sensor, a light sensor, an ambient air temperature sensor, a humidity sensor and an atmospheric pressure sensor.

44. The method according claim 33 wherein the one or more input signals are received from a electrodermal response sensor, a body temperature sensor, an acceleration sensor, a rotation sensor, an orientation sensor, a geolocation sensor and a speed sensor; a sound sensor, a light sensor, an ambient air temperature sensor, a humidity sensor and/or an atmospheric pressure sensor.

45. The method according to claim 33 wherein the one or more input signals comprises analogue sensor time series data discretised and converted into a digital time series prior to performing the method.

46. The method according to claim 33 wherein the one or more input signal is a multi-channel sensor signal combined into a single channel.

47. The method of claim 46 wherein the multi-channel sensor signal comprises a three-dimensional acceleration signal.

48. The method of claim 46 wherein the single channel is a vector length.

49. The method according to claim 33 wherein the circadian rhythm model outputs the cardiac rhythm value as a function of time.

50. The method according to claim 33 wherein the sleep-wake pressure model outputs a sleep-awake pressure value as a function of estimated sleep time and estimated awake time during a previous time period.

51. An apparatus for detecting sleep activity, the apparatus comprising:
an input for receiving one or more input signals;
a processor for processing the input signals;
the processor being configured to execute a method comprising:
performing normalisation of the one or more input signals received by the input;
calculating a circadian rhythm value from a circadian rhythm model and a sleep-wake pressure value from a sleep-wake pressure model;
performing segmentation of the normalised one or more input signals;
calculating a sleep probability value as a function of at least one characteristic of the segmented normalised one or more input signals, the calculated circadian rhythm value and the calculated sleep-wake pressure value; and
outputting an output signal indicating that the one or more input signals comprises a sleep episode, if the sleep probability value is above a predefined sleep detection threshold.

52. A non-transitory computer-readable medium comprising instructions which, when executed by a computer, cause the computer to carry out a method comprising:
receiving one or more input signals;
performing normalisation of the one or more input signals;
calculating a circadian rhythm value from a circadian rhythm model and a sleep-wake pressure value from a sleep-wake pressure model;
performing segmentation of the normalised one or more input signals;
calculating a sleep probability value as a function of at least one characteristic of the segmented normalised one or more input signals, the calculated circadian rhythm value and the calculated sleep-wake pressure value; and
outputting an output signal indicating that the one or more input signals comprises a sleep episode, if the sleep probability value is above a predefined sleep detection threshold.
